(19) United States
(12) Patent Application Publication
(10) Pub. No.: US 2015/0352250 A1
(43) Pub. Date: Dec. 10, 2015

(54) ARTIFICIAL BONE FORMS AND COMPOSITIONS FOR APPROXIMATING BONE

(71) Applicant: NDSU Research Foundation, Fargo, ND (US)

(72) Inventors: Andrew Thomas Dalman, Fargo, ND (US); Michael D. Tchida, Fargo, ND (US); Ross A. Larson, Fargo, ND (US); David L. Wells, Fargo, ND (US); Joel G. Hedlof, Fargo, ND (US)

(21) Appl. No.: 14/830,930

(22) Filed: Aug. 20, 2015

Related U.S. Application Data
(63) Continuation of application No. PCT/US2014/018367, filed on Feb. 25, 2014.
(60) Provisional application No. 61/769,003, filed on Feb. 25, 2013.

(51) Int. Cl. A61L 27/12; A61L 27/16; G09B 23/30; A61F 2/28

(52) U.S. Cl. CPC A61L 27/12; A61F 2/28; A61L 27/16; G09B 23/30; A61L 2430/02

(57) ABSTRACT

An artificial bone construct which approximates one or more components of a skeletal system, wherein the artificial construct is used in the testing and development of new surgical procedures, ballistics testing, implantable devices, etc., and wherein the artificial construct closely approximates the physical and mechanical properties of one or more components of a skeletal system. The artificial bone construct can be implanted in a human body.

### CROSS-REFERENCE TO RELATED APPLICATIONS

This application is a continuation of the PCT/US2014/018367 filed Feb. 25, 2014 and claims priority under 35 U.S.C. §119 to provisional application Ser. No. 61/769,003 filed Feb. 25, 2013, all of which are herein incorporated by reference in their entirety.

### FIELD OF INVENTION

This invention pertains generally to the field of artificial bone forms, tissue and compositions for approximating bone and more specifically to a method and composition for manufacturing and using constructs of artificial bone forms that approximate native tissues, such as for use as a surgical test or practice platform.

### BACKGROUND

There are many experimental situations where approximating the response of human bone to external stimulus is desirable. For example, these could include practice for medical and dental students learning surgical procedures; pre-validation of complex planned surgeries; evaluation of protective helmets and body armor; assessment of injury risk from physical activity. Normally, experiments of this nature would employ cadaver bone or animal bone. Both of these options have significant drawbacks in several operational factors, including extensive administrative overhead, marginal accuracy in replicating intended applications, and limitations on instrumentation and on effectiveness in measuring results.

Dental and medical schools must train future practitioners how to do dental procedures and surgical procedures on realistic materials, that is, materials that closely mimics the actual human bone material on which they will one day be working. Currently available artificial materials and models not only lack realism (in geometry and size) but also do not have the same physical and mechanical properties of actual human bone. This means that medical schools are required to obtain actual human bones, such as human mandibles, in order to have their students practice on something that best approximates the properties of bone. However, obtaining actual human bone material requires special research approval and actual human components are not always in adequate supply.

What is needed in the art is a test bed that closely mimics the geometry, size, and mechanical and physical properties of actual human bone, but which is made from artificial materials that are readily available. In addition, this test bed could be constructed from a material that is biocompatible with the human body, and which could itself be used as a replacement or bone substitute for actual human bone which can be surgically implanted in the body without fear of rejection, and which has a compositional make-up which allows actual growing human bone to integrate into it.

### SUMMARY

Accordingly, one objective of the present invention is to provide a method for the manufacture and use of an artificial bone that closely approximates both the geometry and the properties of native tissue.

It is one objective of the present invention to describe constructs of artificial bone forms to be used in the testing and development of new surgical procedures and implantations.

It is another objective of the present invention to describe a composition which will closely mimic the physical and mechanical properties of native tissue by the materials used to fabricate the artificial construct.

It is yet another objective of the present invention to describe a composition which is bio-compatible with the human body and which can be used as a human bone replacement, such as a bone substitute.

Still another objective of the present invention is to capture digitized data that accurately describes the external and internal geometry, porosity and density configurations of the human bones and applying captured data to create one or more constructs of artificial bone forms.

A still further objective of the present invention is to approximate a bone having a certain density or hardness that transitions to a different density or hardness in an interior portion of the bone to closely approximate similar transitions in real bone.

Further objectives and advantages of the invention will become apparent from a consideration of the drawings and ensuing description.

One exemplary embodiment provides a method for simulating human bone. The method includes, for example, identifying one or more properties of a human bone for simulation, fabricating an artificial bone construct of the human bone based at least in part on the one or more properties, and accounting for one or more intended uses of the artificial bone construct in the fabrication process by adjusting a ratio of one or more constituents of a material composition for replicating the one or more properties of the human bone.

Another exemplary embodiment provides an artificial bone form simulant for human bone. The artificial bone form simulant includes a form characteristic derived from one or more shape properties of human bone and a performance characteristic derived in part from one or more mechanical properties of human bone. One or more of its material constituents include at least one bulk material, binder and curing agent. The ratio of the one or more constituents corresponds to the performance characteristic of the artificial bone form simulant.

Yet another exemplary embodiment provides a method for replicating human bones. First, a human bone for replicating is identified. One or more shape properties and mechanical properties for the human bone are also identified. A material composition is also formulated to account for at least the mechanical properties. The one or more shape properties may be fabricated using the material composition and the material composition may be tailored to account for at least one or more intended uses. Although the steps have an implied order, the present invention contemplates that one or more of the steps prior to fabrication could be performed by varying the order of steps.

### BRIEF DESCRIPTION OF THE DRAWINGS

FIG. 1 is a pictorial representation of a model of one artificial bone form construct in accordance with an illustrative embodiment;

FIG. 2 is a pictorial representation of sample constructs molded from an exemplary artificial bone composition in accordance with an illustrative embodiment;

FIG. 3 is a pictorial representation of a plot comparing a TCP-Bis-GMA in accordance with an illustrative embodiment;

FIG. 4 is a pictorial representation of another plot in accordance with an illustrative embodiment;

FIG. 5 is a pictorial representation of an exemplary artificial bone construct in accordance with an illustrative embodiment;

FIG. 6 is a pictorial representation of an exemplary mold and mold process in accordance with an illustrative embodiment;

FIG. 7 is a pictorial representation of a cross-sectioned pig jaw in accordance with an illustrative embodiment;

### DETAILED DESCRIPTION

This invention pertains generally to the field of artificial bone forms and compositions for approximating bone and more specifically to a method and composition for manufacturing and using constructs of artificial bone forms that approximate native tissues, such as for use as a surgical test or practice platform. For purposes of description, the terms “bone” and “tissue” are used. References to “tissue” are interchangeable with one type of tissue, namely “bone”. For example, the term “tissue” includes at least bone tissue, cartiliganeous bone tissue, and other like bone tissues. At least one object of the invention is the development of an artificial bone construct that approximates native tissue in two crucial ways: 1. Approximation of the geometrical configuration (external and internal) of native bone to high accuracy; 2. Approximation of the physical and mechanical properties of native tissue in the materials used to fabricate the artificial construct.

In one or more aspects, the present invention includes at least the objects of: 1. Capturing digitized data that accurately describe the external and internal geometric configuration of the human bones of interest (e.g., mandible, ribs, sternum, skull, etc.) and applying the captured data to create models that dimensionally-approximate constructs of artificial bone forms; 2. Developing synthetic compositions of material for replicating the physical and mechanical properties of native human tissues; 3. Developing processing methods for reliably and efficiently fabricating artificial constructs that approximate human bone.

Experimental:

At least all of the intended objectives of the present invention are achieved in the following exemplary constructs of artificial bone forms. Although specific human bones and tissue are addressed, the following is intended to set forth exemplary and experimental processes that could be applied to any intended approximation of human bone and tissue. Specific bone and tissue references are for the purpose of illustrating that the present invention meets at least all of its intended objectives.

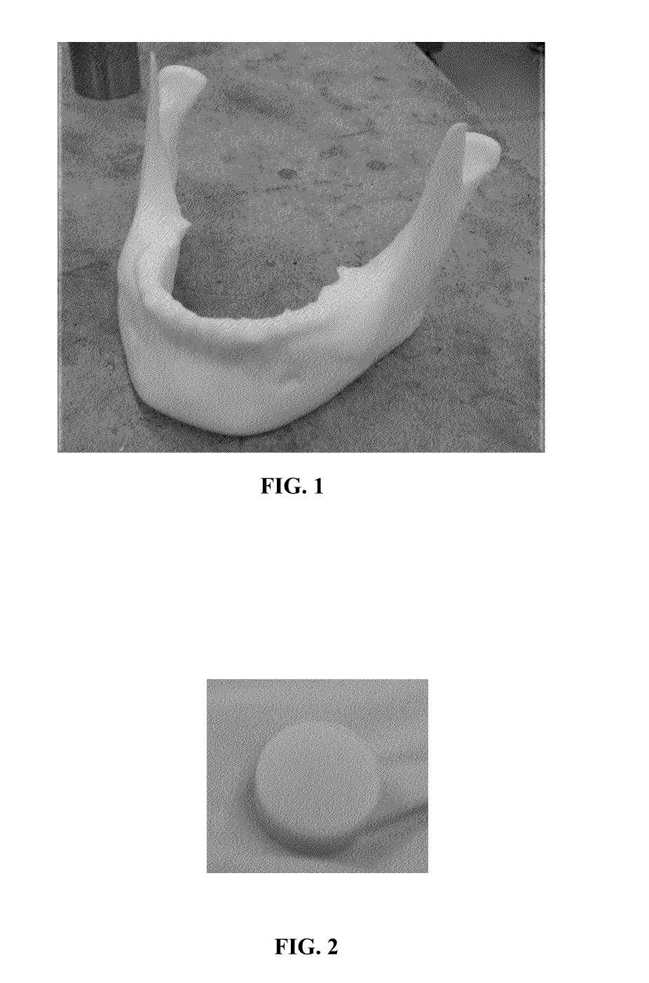

In one aspect, the present invention includes the development of an artificial jaw, with a focus and research concentrated on the human mandible. There are two distinct bone types, such as in the mandible. These include, for example, cortical bone which comprises the hard outer shell of the jaw and trabecular (or cancellous) bone which comprises the porous, softer inner core. Data representative of the mandible of an average forty-year-old male may be digitized and converted to a three-dimensional solid model. The data may be used and applied, for example, in printing a full-scale, dimensionally-accurate model of a human mandible as pictorially represented in FIG. 1.

The printed mandible model shown in FIG. 1 may be fabricated from an acrylonitrile-butydiene-styrene (ABS) polymer using, for example, three-dimensional printing (often referred to as additive manufacturing). Thus, from at least this process, external bone geometries can be approximated in a polymeric substance. Therefore, another object of the present invention is to approximate internal bone geometries and provide processing methods for producing the same geometries from materials that match properties of human bone.

In one embodiment, an artificial mandible may be used for the testing and prototyping of surgical procedures and implants. In alternate embodiments, an artificial mandible could be designed for implantation in the human body as a mandible replacement or implant, without varying from the intent of the invention as described herein. It should also be noted that the composition described herein can be used for bone replacement other than the mandible, and that no limitations should be assumed or are implied by the examples herein regarding the human mandible, which are provided simply by way of at least one example of the present invention.

Experimental Fabrication:

According to a preferred exemplary aspect of the present invention, constructs of artificial bone forms may be fabricated by direct 3D printing of the final shape from a set of simulant materials. Modifications of commercially-available additive manufacturing equipment could be made to accommodate, if needed, a new set of deposition materials. Another exemplary fabrication method employs the identical digital data used, for example, in 3D printing an ABS jaw construct, but in the reverse, by producing a cavity of the shape of the bone, inside of an outer shell. The cavity, then, can be used for molding the bone shape. This is a multi-step exemplary process. First, a cavity may be used to mold a cortical shell from the artificial material. A second cavity configuration may then be produced from which a trabecular reproduction can be molded. The two bone components may then be assembled to complete the artificial bone construct. In this process, the cortical shell of native bone completely encloses the trabecular core. Also in this approach, 3D printing could still be accomplished using ABS, as the printed object in this instance is the tooling piece, rather than the finished artificial bone construct.

According to another exemplary aspect of the invention, a synthetic rib cage may be developed as another one of the many constructs of artificial bone forms contemplated by the present invention. In this instance, for example, the torso bone contains two types of osseous tissue, namely cortical and trabecular, but in a different configuration from a mandible construct. Geometric approximation of rib cage bones begins with collection of physiological data that describe ribs and sternum of adult humans. Data may then be captured in digital form in a robust computer-aided-engineering environment. From this digital foundation, 3D printing of bone forms by either positively or negatively rendering can be accomplished in the same manner as a mandible.

Although at least one fabrication process is set forth above, the present invention contemplates numerous other viable and exemplary types of processes for fabricating constructs of artificial bone forms in accordance with one or more objects of the present invention. For example, various forms of additive processing using a digital model may be used, such as for example, 3D printing, ceramic printing, light curing (e.g., Digital Light Processing (DLP), Laser Additive Manufacturing (LAM), or Stereolithography (SLA)). According to other exemplary processes, one or more molding processes may be used for fabricating constructs of artificial bone forms in accordance with one or more objectives of the present invention. For example, injection molding (chemical or microwave cured), rotational molding (chemical or microwave cured), chemical cure (e.g., chemical reaction forming bubbles that establish a trabecular structure, such as a heat catalyst initiating the cure), and dip and cure (e.g., make internal structure, dip it in cortical compound to coat it, take it and cure it layer by layer like a candle—post processing operations may not be needed to obtain a final construct geometry). According to still other exemplary processes, one or more subtractive processes may be used for fabricating constructs of artificial bone forms in accordance with one or more objects of the present invention. For example, CNC milling, wax burnout (e.g., primarily for internal trabecular structure), and negative shell forming (e.g., possibly a complete negative of the bone construct (internal and external) pour into a cast and then remove wax mold).

Material Approximation:

Important properties of human bone to be approximated in an artificial bone construct of the present invention are those of mechanical strength, namely tensile strength, compressive strength and elastic modulus. Other mechanical properties of interest include, for example, shear strength and flexural modulus. In some instances, one or more mechanical properties may be more important for obtaining a set of design properties. By way of example, for the purposes of application of artificial bone in experiments in ballistic effects, fatigue strength is generally deemed not as important. However of greater importance in this particular instance (e.g., ballistics testing) is the physical property of specific gravity, as the density of an artificial bone construct will likely have an influence on the correlation of experimental data to actual personnel effects. For purposes of the clarification, specific gravity is a ratio is better understood as a ‘relative density’ characteristic. By definition it is the ratio of the density of a substance (e.g., bone and bone tissues) to the density of water @4° C. Bone is described as a connective tissue, helping to support and bind together various parts of the body. Bone is a composite material consisting of both fluid and solid phases. Its specific gravity is about 2.0 but varies depending on the type of bone. Density on the other hand is a mass per volume metric and porosity is typically expressed as a percentage or fraction and defines the amount of empty space in a material.

Reliable properties of human bone tissue are somewhat difficult to state with certainty given their variability due to age, health and size. Bone densities can vary whether the human bone of interest and mimic is healthy or suffers from osteoporosis, such as from a calcium deficiency. As such, reported data may vary considerably. Bone, as with most ceramic materials, is stronger in compression than in tension. Both compressive and tensile strength are significantly higher than shear strength. Bone is noticeably anisotropic, with longitudinal and transverse values of tensile strength in long bones (e.g., femur) differing by an amount from 50 to 100 percent. Likewise, human bone is somewhat viscoelastic; thus, as strain rate increases, so do tensile strength and elastic modulus. Most data available are for strain rates common to standard tensile testing machines, ranges, in order, from about 0.001 to 1 in/in/sec. There is evidence that at the high strain rates relevant to ballistic response, ultimate tensile strength and elastic modulus are significantly higher and failure strain is substantially lower. It is also noteworthy that reported values of tensile strength of the trabecular structure are on the order of 5 percent of those for cortical bone.

TABLE 1
Target Mechanical Properties of Cortical Bone

| | Target Value | | Range | |
|---|---|---|---|---|
| compressive strength | | | | |
| longitudinal | 200 | MPa (29.0 ksi) | 70-280 | MPa |
| transverse | 50 | MPa (7.3 ksi) | ~50 | MPa |
| tensile strength | | | | |
| longitudinal | 135 | MPa (19.5 ksi) | 50-150 | MPa |
| transverse | 50 | MPa (7.3 ksi) | ~50 | MPa |
| shear strength | 75 | MPa (10.9 ksi) | ~75 | MPa |
| elastic modulus | | | | |
| longitudinal | 17.4 | GPa (2.5 × 10⁶ psi) | 11-21 | GPa |
| transverse | 9.6 | GPa (1.4 × 10⁶ psi) | 5-13 | GPa |

Representative values for strength of long bones (e.g., the femur) are shown above in Table 1. As a starting position, the targets to be approximated by the artificial material may be taken as the strength values for a longitudinal orientation of long bones. For example, specific gravity of cortical bone is reported in the range of 1.0 to 1.9, with the most common value at 1.6.

Human bone is comprised of a form of calcium phosphate called hydroxyapatite (HAP), namely Ca₁₀(PO₄)₆(OH)₂. HAP is a synthesized product that is readily available from commercial suppliers. Similar compounds may also be used, such as for example, tricalcium phosphate (TCP), namely Ca₃(PO₄)₂ and alkali-substituted calcium phosphates, such as for example, CaNaPO₄, CaKPO₄ and Ca₂KNa(PO₄)₂. Also of interest in replicating properties of native bone are one or more oxides being used as prospective material components. For example, one or more oxides may include alumina, namely Al₂O₃ and cristobalite, a polymorph of silica, namely SiO₂. Other examples of oxides include zirconia, namely ZrO₂.

According to one approach, a composite material is composed using a stable thermoplastic binder with a ceramic powder hard phase. Although specific materials and material compositions are provided, the object is a composite material that is moldable or printable into the shapes necessary to emulate the geometry of an artificial bone construct to be simulated and where the ceramic hard phase will provide the mechanical properties that mimic those of native bone. According to one method, the hard phase is comprised of mixture of HAP and cristobalite. Other considerations include TCP as more suitable for these purposes and alumina as a more suitable oxide contributor.

According to one exemplary process, a binder of bisphenol A-glycidyl methacrylate (bis-GMA) may be used. This binder is a polymeric compound frequently used in dental prostheses, usually with a filler such as silica or various proprietary glasses for both wear resistance and appearance. In clinical dental applications, bis-GMA may be used in a mixture that is cured by ultraviolet light. According to another exemplary process, the polymer may be treated with a photo-initiator (such as camphorquinone (CQ), phenylpropanedione (PPD) or lucirin (TPO)) and an additive (such as dimethylglyoxime) to help control flow characteristics. A catalyst may also be used to control reaction rates.

TABLE 2
Exemplary Materials for Artificial Bone Constructs

| Formation | | Bioactive | Reaction | Inorganic |
|---|---|---|---|---|
| Process | Polymers | Glasses | Initiators | fillers |
| Ultraviolet | Bis-GMA, | SiO₂, Na₂O, | camphorquinone, | hydroxyapatite, |
| Light | PEEK, | CaO, P₂O₅, | phenyl- | tricalcium |
| Curing | TEDGMA, | Al₂O₃ | propanedione, | phosphate, |
| Process | PHB, | | lucirin, | alkali- |
| | PDLLA, | | dimethyl- | substituted |
| | UDMA, | | aminoethyl | calcium |
| | HDDMA | | methacrylate | phosphates |
| Microwave | Bis-GMA, | Potentially | benzoyl peroxide | Potentially could |
| Curing | TEDGMA, | use the same | | use the same as |
| Process | | as UV light | | UV light process |

Although an option, cure initiation by ultraviolet light exposure may not be fully suitable. Experiments employing the same ultraviolet light source as is used in clinical dental applications revealed that the effective penetration of ultraviolet rays may be only on the order of 1 to 2 millimeters. The section thicknesses required to approximate all but the smallest bones are generally too thick for effective penetration of light to affect complete cure of the polymer. Thus, effective curing of thicker sections may than be possible by means of a layered application of the composite. Although ultraviolet curing is an option, particularly with alternative procedural variants, other approaches are also contemplated. One intended composition, namely Bis-GMA and TEDGMA, may be cured using either a microwave or UV light curing process. Moreover, PEEK may also be a viable option for UV light curing. Other exemplary polymers for forming one or more artificial bone constructs may include PHB and PDLLA, which are biodegradable polymers used in scaffold engineering. Such polymers may be UV light resistant and thus a viable option.

TABLE 3
Exemplary Polymeric Materials for Artificial Bone Constructs

| Polymer | Brief Description |
|---|---|
| Bis-GMA | Common dental restorative polymer that |
| | serves as an organic matrix |
| PEEK | Thermoplastic organic polymer that has the potential |
| | to represent shape memory polymer characteristics |
| TEDGMA, | Another type of polymer used as dental restorative. |
| UDMA, | In addition makes Bis-GMA easier to work with |
| HDDMA | |
| PHB | A thermoplastic polymer produced by many types of |
| | microorganisms that is biodegradable. Has |
| | piezoelectric properties that stimulate bone growth. |
| PDLLA | Typically used in engineering scaffolds for both |
| | hard and soft tissue development |

TABLE 4
Exemplary Material Abbreviation Descriptions

| Abbreviation | Full Name |
|---|---|
| Bis-GMA | bisphenol A-glycidyl methacrylate |
| TEDGMA | triethylene glycol dimethacrylate |
| PEEK | polyether ketone |
| PHB | poly(3 hydroxybutyrate) |
| PDLLA | poly-DL-lactide |
| SiO₂ | crystobalite |
| UDMA | urethane dimethacrylate |
| HDDMA | hexamethylene dimethacrylate |
| Na₂O | sodium oxide |
| CaO | calcium oxide |
| Al₂O₃ | alumina |
| P₂O₅ | diphosphorus pentoxide |

Although specific materials are referenced, Tables 2-4 provide additional exemplary materials for composing a material for forming one or more constructs of artificial bone forms.

By way of further example, a microwave cure process may be employed for curing artificial bone constructs. The required microwave frequencies and energy level are comparable to those used in commercial home food preparation devices. In the case of microwave cure of the polymeric composite, an initiator of benzoyl peroxide (BPO), namely C₁₄H₁₀O₄, may be used.

According to another aspect of the present invention, curing is used to approximate various bone tissue types. For example, over or under curing of selected areas of an artificial bone form may be performed to produce different mechanical properties (in a gradient, specific sections, etc.) within the same artificial bone form. Thus, an artificial bone form, such as a rib cage, could be fabricated as a single, unitary (e.g., a model in one piece), in one process, with one material by controlling the cure of various sections relative to other sections of an artificial bone form such as a ribcage. For example, more flexible areas in an artificial bone form may be realized by varying the cure of one localized or targeted area of an artificial bone form relative to another localized or targeted area of the same artificial bone form. In at least one instance, more flexible areas in a single artificial bone form may be engineered to approximate, for example, cartilaginous bone.

An exemplary composition process includes molding of small buttons of prospective composite formulations for an artificial bone construct as illustrated pictorially in FIG. 2. Teflon molds may be used for buttons, such as ones formed to have dimensions of 3 millimeters thick by 6 millimeters in diameter. After curing, the compositions may be tested for hardness, as a first screen of the desirable mechanical properties. Hardness is somewhat proportional to tensile strength and serves as a convenient mechanical property for screening purposes. Experimental variables include both material component proportions and curing process parameters. Test results indicate at least that the proposed materials are feasible for fabricating artificial bone constructs, and achieving the desired properties through control of composite components and processing. By controlled variation in composite composition and processing parameters a bone simulant (i.e., artificial construct) may be produced to match native bone characteristics of a varying population.

When the ranges of potential compositions and curing parameters have been narrowed, samples necessary to refine the composite and to more completely characterize physical and mechanical properties are produced. The physical property of primary interest is specific gravity. While it is not believed that artificial bone must have the precise same overall density as the native tissue for effective practical emulation, it is felt that correlation of data derived from impact studies would be enhanced by a close approximation. Mechanical properties are more important in governing response behaviors. The objective is that the artificial bone produced will very closely approximate the dynamic behavior of native tissue, which in engineering terms is described through mechanical properties. Mechanical properties of primary interest are tensile and compressive strength and modulus of elasticity as indicated above.

Prototype Artificial Bone Forms:

One or more prototype artificial bone forms may be produced, these may include various human bones of interest, such as for example, an artificial rib and artificial sternum bones, which may be assembled together into an emulated rib cage. For example, one or more artificial bone constructs could be assembled together with an adhesive selected to have, for example, characteristics and parameters replicating cartilaginous tissue. This could include using a silicone adhesive, such as those available through Nu-Sil Technology. According to another exemplary method, the artificial bone form could be fabricated by direct printing whereby the entire rib-sternum assembly could be printed as one complete artificial bone construct. The tooling devised in the geometric approximation work is preferably married with a composite composition developed in the material approximation effort. Samples of close-bone forms are produced, employing tooling devised in the geometry leg of the research, using composite compositions and curing methods developed in material processing.

Commensurate with the following description, at least one or more objects of the present invention are directed at establishing: 1. The proportions of components that will result in the best match to target properties in the resulting composite material; and 2. A set or range of processing parameters necessary to produce reliable constructs of artificial bone forms.

Analysis:

Because an artificial bone form such as a jaw will share the same shape and material strength as a real human jaw, it should react in the same manner as a human jaw for a given stress placed upon it. The same is true and preferred for any human bone mimicked herein by one or more fabricated artificial bone forms. For purposes of illustration and discussion only, what follows is an analysis of one construct of an artificial bone in the form of a jaw bone or mandible.

The jaw is being designed so that it will mimic the human jaw, as closely as possible. In should be noted that variations on the composition (which do not go beyond the intent of the present invention) can be made such that artificial jaws of varying density and other mechanical properties can be made, such that the mechanical properties of a wide range of human bone types can be mimicked, from young, healthy bone to the bones of someone suffering from osteoporosis.

In accordance with an exemplary aspect of the present invention, a computer model of a human jaw is created and used. The model may be used to develop a negative model to function as a casting mold for the jaw.

A demonstration of this concept is currently available in the form of a rapid-prototyped model of a portion of a mandible. While the current sample is made of ABS plastic instead of ceramic composite (as is the intended final composition), it showcases the structure and features of the jaw design.

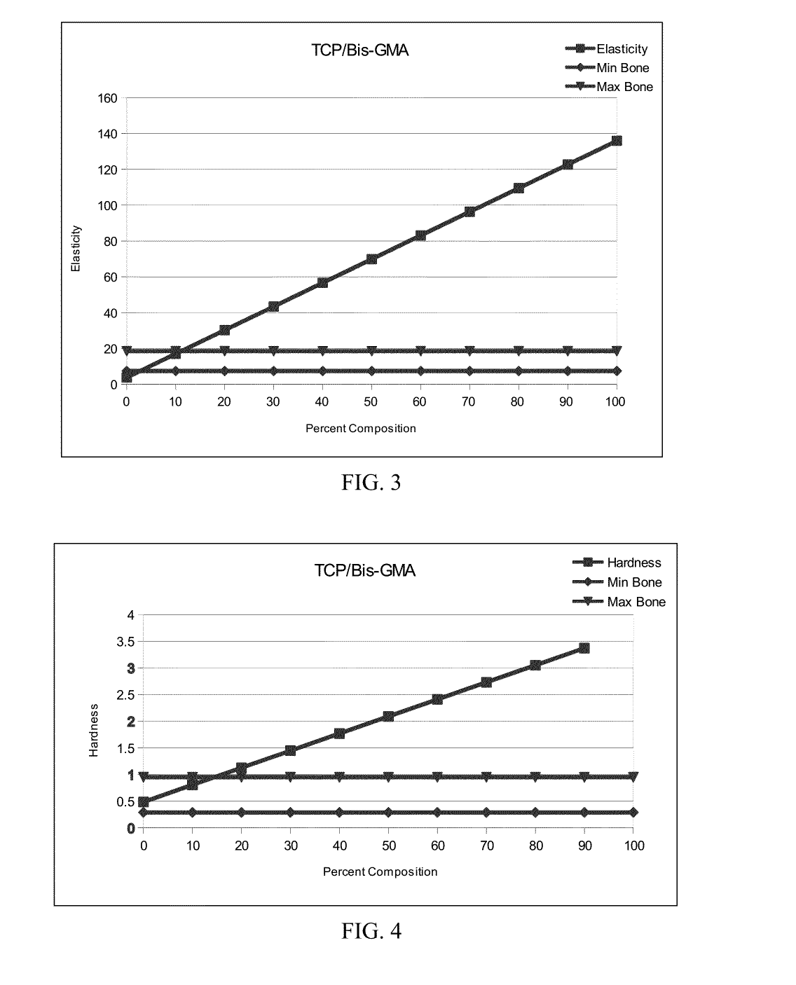

According to one exemplary process, a primary construction material for the jaw may be a ceramic composite. Included in this composite may be a binder, a core material, and a curing agent. Hydroxyapatite (HAP) has been shown to have similar properties to human bone and may be used as the core material in one embodiment of the invention, although any similar, appropriate material, such as the materials set forth above at least in Tables 2-4, may be used in alternate embodiments. A resin such as BIS-GMA (bisphenol A glycidyl methacrylate), which is used in dental procedures involving the replacement of enamel and the sealing of cavities and binds with a core material to produce a hard yet flexible composite, may be used by varying the properties to alter the ratio of the mixture in accordance with one or more preferred compositions as pictorially represented in FIG. 3.

Note that these graphs are comparing a TCP-Bis-GMA mix and not an HAP/Bis-GMA mix (TCP is tricalcium phosphate). Extensive research has been put in to determining the properties of HAP and it has been found that TCP and HAP share similar properties and are both close to human bone. Note that the elasticity is in GPa (gigapascals) and Percent Composition is a ratio of TCP to Bis-GMA.

As the graph shown in FIGS. 3-4 illustrate, human bone elasticity rests in the 7 to 19 Gpa range. In order to create a mixture within these constraints, embodiments of the present invention contemplate ranges having a 4 to 15 percent ratio of HAP/TCP to Bis-GMA as a possible composition for fabricating artificial bone forms in accordance with the present invention. The mixture ratio can be altered based on the results of the tests to achieve a composition that achieves one or more of the objectives of the present invention.

Human bone has an average hardness of 0.29 to 0.95 GPa. In order to approximate this, the HAP/TCP to Bis-GMA ratio is preferably between 0 and 15%.

When the limitations of both graphs shown in FIGS. 3-4 are taken into account, a range of 4-15% HAP/Bis-GMA may be a preferred ratio for purposes of testing a construct. A construct material composition may be selected from this range (tentatively 5%, 10%, and 15%) for testing, and specifically for obtaining the parameters set forth in Table 1 above.

The following materials may be used in the creation of the artificial bone material, in the amounts listed. Listed amounts are what are required to manufacture one ‘unit’ of resin (non-solids) master mix material. Solids are then added to this material in order to bring it up to the required % by mass solids composition to achieve the required mechanical properties. One exemplary composition may include 1 g BIS-GMA (2,2-bis[4-(2-hydroxy-3-methacrtyloxproxy)phenyl]-propane), 0.43 g TEG-DMA (triethylene glycol dimethacrylate), 0.014 g camphorquinonine, 14.28 microliter ethyl 4-dimthylaminobenzoate, which is equivalent to roughly 1.457 g total. The solid content may then be determined by the equation: 1.457/x−1.457; x=100−% comp, where x=50% cristobalite, 50% hydroxyapatite, by mass. Exemplary mixing ratios are shown below in Table 5.

TABLE 5
Exemplary Mixing Ratios

| | | Hydrodyapatite | Crystobalite |
|---|---|---|---|
| For control: | no solid | 0 | 0 |
| NO filler | | | |
| For 5%: .0766 g | .038 g each solid | 0.038 | 0.042 |
| solid mix | | | |
| For 10%: .1612 g | .081 g each solid | 0.083 | 0.082 |
| For 15%: .257 g | .129 g each solid | 0.133 | 0.13 |
| For 20%: .364 g | .182 g each solid | 0.179 | 0.181 |
| For 25%: .486 g | .243 g each solid | 0.241 | 0.241 |
| For 30%: .624 g | .312 g each solid | 0.314 | 0.311 |
| For 35%: .784 g | .392 g each solid | 0.391 | 0.393 |
| For 40%: .972 g | .485 g each solid | 0.482 | 0.483 |

An exemplary composition may be prepared by massing all the materials. If the total mass of sample is to be a certain number of grams, the ratio of the mix materials may be determined to deduce how many grams of each material will be required. The BIS-GMA is heated to 75 degrees Celsius to become workable and visible clumps are removed. A curing mold is filled, for example, ⅓ of the way full and the layer of mixture is cured using UV light for roughly 1-2 minutes. The filling and curing steps may be repeated until all the material is used. In another curing process, microwave curing may be used to cure the mixture. The microwave curing process substitutes camphorquinonine and crystobalite for benzoyl peroxide. It serves the dual-purpose of oxide and initiator in the microwave curing process. Samples may be cured for 5 minutes @ 2.45 GHz in a 700 Watt consumer microwave. Samples are allowed to sit for 30 minutes at room temperature after cure and allowed to age for one full day before testing.

Configuration:

The current conceptual configuration is in the shape of a human mandible. It consists of a solid shell with a hollow core, emulating the histology of a human mandible as closely as possible in terms of shell thickness variation and strength.

A fleshy outer layer is included in the design concept, as well. It will consist of a material that approximates the nature of the gums. A gelatin mix known as ballistics gel, predictably used in ballistics testing, mimics the properties of human tissue and may be used for this coating, although any appropriate material may be used without varying from the intent of the present invention.

An image of an artificial human mandible shown in FIG. 5 is included for reference. Note that the design does not include teeth at this point.

Process Engineering:

A mixture may be molded and cured with Ultraviolet light as discussed above. A photoinitiator sensitive to a wavelength between 380 and 515 nanometers may be used to allow the use of UV light in the curing process, which should not have an effect on the mechanical properties of the composite.

According to one method, a composite mixture is placed into a translucent mold and bombarded with ultraviolet light of a desirable wavelength. The artificial jaw construct is carefully removed after curing in order to prevent damage to the design or the mold. One or more controlling factors in this method are the strength of the emitted light and the time of exposure. Opacity of the mold and uniformity of curing are also accounted for during the curing process.

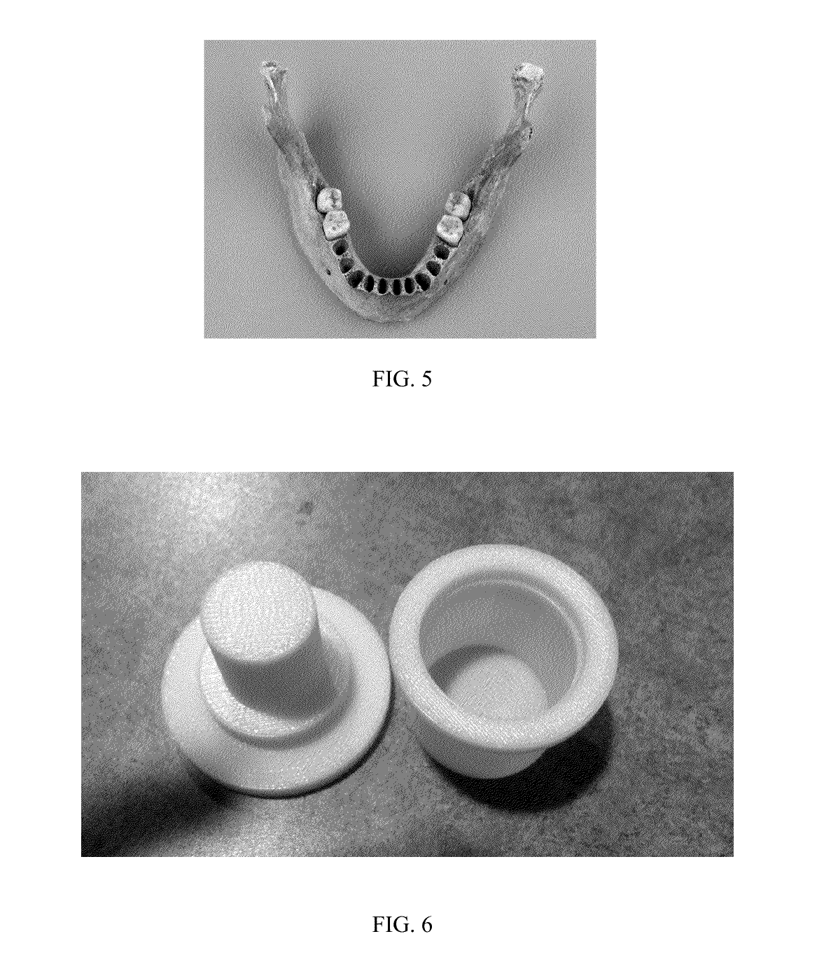

An image of an exemplary molding cup designed to contain the mixture during curing is shown in FIG. 6. The protrusion on the left serves to insure the material remains hollow wherein the finished design is intended to approximate a hollow cylinder.

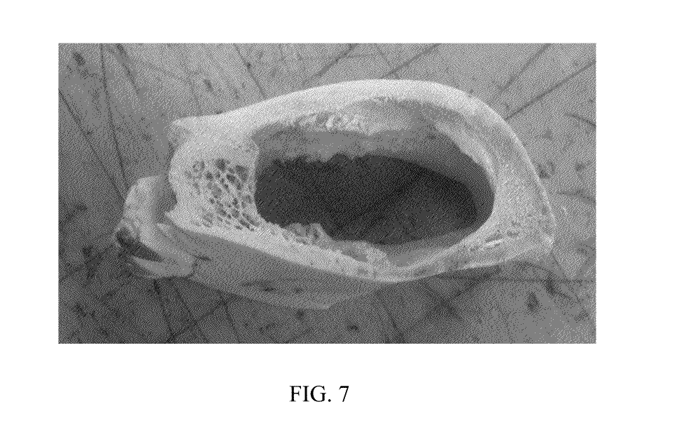

FIG. 7 provides an image of a sectioned pig jaw illustrating the differentiations between hard (cortical) and soft (trabecular) bone (e.g., ratio of cortical to trabecular). Also note the inconsistency in the thickness of the bone shell (e.g., longitudinal versus transverse). The present invention contemplates tuning the composition to mimic different bone types, densities, health, etc. For example, an artificial bone construct composition can be tuned to account for any of the above-identified parameters. Soft trabecular bone could be formed from a separate composition from a hard cortical bone composition so that each exhibits and mimics properties of an approximated human bone. The two could be combined using any one of the applicable processes set forth above, such as through adhesive bonding. Depending on the formation process, one layer could be formed/grown from another layer to account for the difference in the ratio of cortical to trabecular bone, or the ratio of change in the thickness of one layer either transversely or longitudinally. In this manner, compositions for various artificial bone constructs may be prepared for simulating by approximating various human bones. Simulations' focus lies in the mechanical properties obtained from the compositions prepared for replicating artificial bone constructs. The accuracy of simulation may rely on empirical testing of various mixture ratios to determine how closely the approximation mimics the documented properties and behavior of human bone.

In another aspect, simulation of one or more constructs of artificial (i.e., human) bone forms addresses either or both of the macro- and micro-mechanical properties of the bone for emulation in accordance with a preferred end-use. For example, for surgical emulation the micro-mechanical properties of the artificial bone construct are weighted heavier than the macro-mechanical properties. Used in testing and development of new surgical procedures and implantations, the micro-mechanical properties may be given greater weight in the approximation process. Conversely, use of an artificial bone construct for ballistics testing the macro-mechanical properties may be given greater emphasis in the approximation process. In this manner, the formulations and cure parameters may be specifically tuned to achieve a set of preferred macro- or micro-mechanical properties.

Having described the preferred embodiment, it will become apparent that various modifications can be made without departing from the scope of the invention as defined in this document. In particular, alternate materials than those described herein may be used in place of those described without departing from the scope of the invention, as long as the alternate materials allow the same mechanical and physical properties of human bone to be mimicked.

Also, variations on the ratios of material used beyond those described herein may be used. In fact, it may be desirable to “tune” the physical characteristics of the material to mimic different bone densities, so that medical and dental students are trained on materials closely mimicking the densities of people of all ages, health, and size.

Although the examples in this specification primarily cover an implementation that represents a human mandible bone, the described composition and methodology can be used in any type of human bone.

The composition as described is bio-compatible and may be optimized for use as a replacement of human bone for implantation in the human body.

### REFERENCES

All the references as listed below are herein incorporated by reference in their entirety.
1. N. P. Piesco, Histology of dentin, in: J. K. Avery (Ed.), Oral Development and Histology, Thieme Medical Publishers Inc., New York, 1994.
2. “Sintering properties of hydroxyapatite powders prepared using different methods”, http://dx.doi.org/10.1016/j.ceramint.2012.05.103
3. Biomatlante corporation homepage, http://www.biomatlante.com/products
4. “Something to chew on: How do we actually test dental materials?”, http://www.health.umn.edu/healthtalk/2012/08/16/something-to-chew-on/
5. Fackerl, Martin L Effects of small arms on the human body. Letterman Army Institute of Research, California.
6. “An improvement in sintering property of β-tricalcium phosphate by addition of calcium pyrophosphate”, School of Materials Science & Engineering, College of Engineering, Seoul National University, Shinrim-dong, San 56-1, Kwanack-Ku, Seoul 151-742, South Korea, http://www.sciencedirect.com.proxy.library.ndsu.edu/science/article/pii/S0142961201002010
7. “Antimicrobial and physicochemical properties of experimental light curing composites with alkali-substituted calcium phosphate fillers”, Department for Functional Materials in Medicine and Dentistry, University of Würzburg, Pleicherwall 2, 97070 Würzburg, http://www.sciencedirect.com.proxy.library.ndsu.edu/science/article/pii/S0109564112000218

What is claimed is:

1. A method for simulating human bones comprising:
identifying one or more properties of a human bone for approximating the human bone;
fabricating an artificial bone construct of the human bone based at least in part on the one or more properties; and
accounting for one or more intended uses of the artificial bone construct in the fabrication process by adjusting a ratio of one or more constituents of a material composition for replicating the one or more properties of the human bone.

2. The method of claim 1 wherein the one or more constituents comprise a binder, a bulk material and a curing agent.

3. The method of claim 1 wherein the one or more properties comprise a ratio of cortical bone to trabecular bone.

4. The method of claim 1 wherein the one or more properties comprise a ratio of change in bone density along at least one transverse and/or longitudinal direction.

5. The method of claim 2 wherein the binder comprises bisphenol A glycidyl.

6. The method of claim 1 wherein the one or more intended uses comprise at least one of:
a. a bone-destructive process;
b. a bone-interrogative process;
c. a bone replacement process.

7. An artificial bone form simulant for human bone, comprising:
a form characteristic derived from one or more shape properties of human bone;
a performance characteristic derived in part from one or more mechanical properties of human bone;
one or more material constituents comprising at least one bulk material, binder and curing agent; and
a ratio of the one or more constituents corresponding to the performance characteristic.

8. The artificial bone form simulant of claim 7 further comprising a bonding adhesive replicating cartilaginous tissue, wherein the bonding adhesive is between a pair of the artificial bone form simulants.

9. The artificial bone form simulant of claim 7 further comprising a bonding adhesive between a pair of bone types.

10. The artificial bone form simulant of claim 7 wherein the ratio of the one or more constituents corresponds at least in part with bone type comprising at least one of:
a. cortical bone;
b. trabecular bone.

11. The artificial bone form simulant of claim 7 wherein the binder comprises bisphenol A glycidyl.

12. The artificial bone form simulant of claim 7 wherein the bulk material comprises hydroxyapatite.

13. The artificial bone form simulant of claim 7 wherein the mechanical properties correspond at least in part to bone densities derived from human bone.

14. A method for replicating human bones, comprising:
identifying a human bone for approximating;
acquiring one or more shape properties and mechanical properties for the human bone;
formulating a material composition accounting for at least the mechanical properties;
fabricating the one or more shape properties with the material composition; and
tailoring the material composition to account for at least one or more intended uses.

15. The method of claim 14 wherein the tailoring step includes altering the ratio of the material composition associated with cortical bone type and trabecular bone type.

16. The method of claim 14 wherein the mechanical properties comprise one or more macro-mechanical and micro-mechanical properties associated with the one or more intended uses.

17. The method of claim 14 wherein the step of fabricating comprises one or more material additive processes.

18. The method of claim 14 wherein the one or more intended uses comprise at least one of:
a. a bone-destructive process;
b. a bone-interrogative process;
c. a bone replacement process.

19. The method of claim 14 wherein the one or more mechanical properties comprise a ratio of change in bone density along at least one transverse and/or longitudinal direction.

20. The method of claim 14 further comprising controlling curing of the material composition based at least in part of the one or more mechanical properties.
